(19) United States
(12) Patent Application Publication
(10) Pub. No.: US 2006/0155351 A1
(43) Pub. Date: Jul. 13, 2006

(54) Cooling blanket assembly

(76) Inventor: Matthew C. Matson, Edmonds, WA (US)

Correspondence Address: SEAN KAUFHOLD, P. O. BOX 89626, SIOUX FALLS, SD, 57109, US

(21) Appl. No.: 11/032,597

(22) Filed: Jan. 11, 2005

(51) Int. Cl. A61F 7/00

(52) U.S. Cl. 607/114

(57) ABSTRACT

A cooling blanket assembly includes a substantially waterproof and flexible housing having a top wall, a bottom wall and a peripheral wall extending between and attached to the top and bottom walls. The housing has an inner surface an outer surface. The housing has a substantially rectangular shape with a length generally between 3 feet and 5 feet and a width generally between 3 feet and 5 feet. A plurality of ammonium nitrate crystals is positioned within the housing. A plurality of containers is positioned within the housing. Each of the containers has water therein. At least one rigid plate is attached to the inner surface of the top wall. The containers may be selectively broken on the plate such that the water contained therein saturates adjacently positioned portions of the ammonium nitrate.

### BACKGROUND OF THE INVENTION

1. Field of the Invention

The present invention relates to cooling devices and more particularly pertains to a new cooling device for cooling down a person suffering from hyperthermia.

2. Description of the Prior Art

The use of cooling devices is known in the prior art. U.S. Pat. No. 3,429,315 describes a blanket including upper and lower chambers separated by each other by a clamp. When the clamp is opened, the contents of the upper and lower chambers are allowed to mix so that a endothermic reaction occurs. Another type of cooling device is U.S. Pat. No. 6,233,945 which includes a cold pack having a pair of chambers therein. The chambers again have material therein that when mixed produce an endothermic reaction.

While these devices fulfill their respective, particular objectives and requirements, the need remains for a blanket having a construction which is easier to use and store than the devices found in the prior art.

### SUMMARY OF THE INVENTION

The present invention meets the needs presented above by generally comprising a substantially waterproof and flexible housing having a top wall, a bottom wall and a peripheral wall extending between and attached to the top and bottom walls. The housing has an inner surface an outer surface. The housing has a substantially rectangular shape with a length generally between 3 feet and 5 feet and a width generally between 3 feet and 5 feet. A plurality of ammonium nitrate crystals is positioned within the housing. A plurality of containers is positioned within the housing. Each of the containers has water therein. At least one rigid plate is attached to the inner surface of the top wall. The containers may be selectively broken on the plate such that the water contained therein saturates adjacently positioned portions of the ammonium nitrate.

There has thus been outlined, rather broadly, the more important features of the invention in order that the detailed description thereof that follows may be better understood, and in order that the present contribution to the art may be better appreciated. There are additional features of the invention that will be described hereinafter and which will form the subject matter of the claims appended hereto.

The objects of the invention, along with the various features of novelty which characterize the invention, are pointed out with particularity in the claims annexed to and forming a part of this disclosure.

### BRIEF DESCRIPTION OF THE DRAWINGS

The invention will be better understood and objects other than those set forth above will become apparent when consideration is given to the following detailed description thereof. Such description makes reference to the annexed drawings wherein:

FIG. 1 is a perspective view of a cooling blanket assembly according to the present invention.

FIG. 2 is a top view of the present invention.

FIG. 3 is a cross-sectional view taken along line 3-3 of FIG. 1 of the present invention.

FIG. 4 is a cross-sectional view taken along line 4-4 of FIG. 2 of the present invention.

### DESCRIPTION OF THE PREFERRED EMBODIMENT

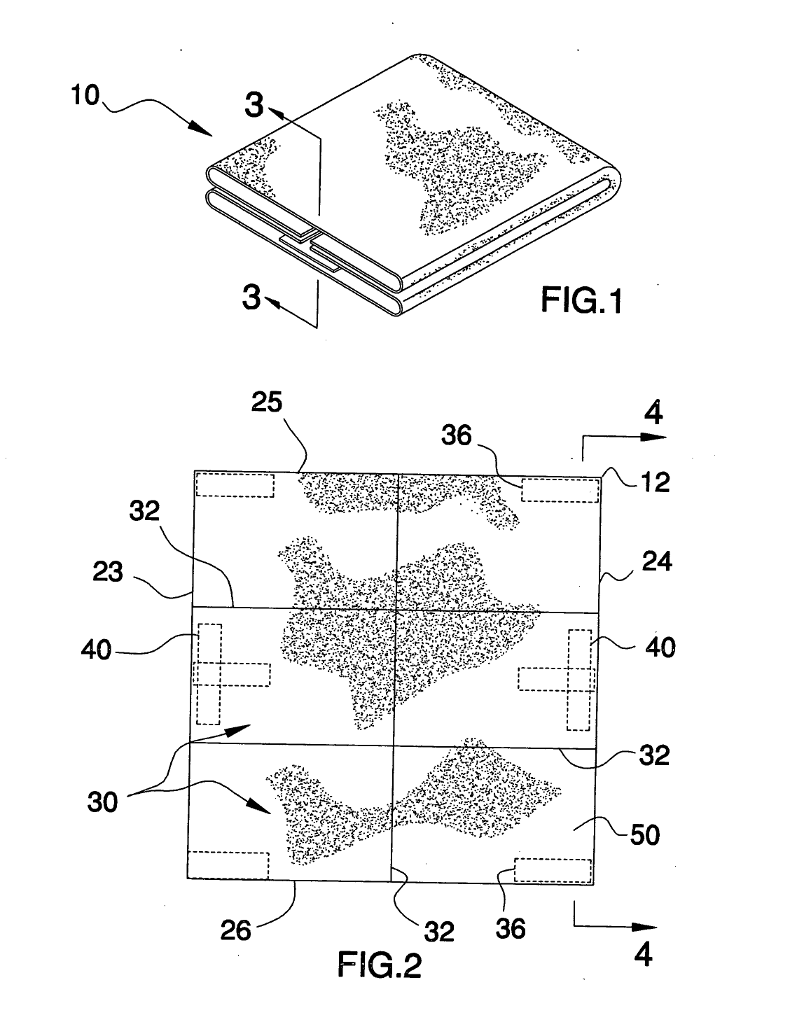

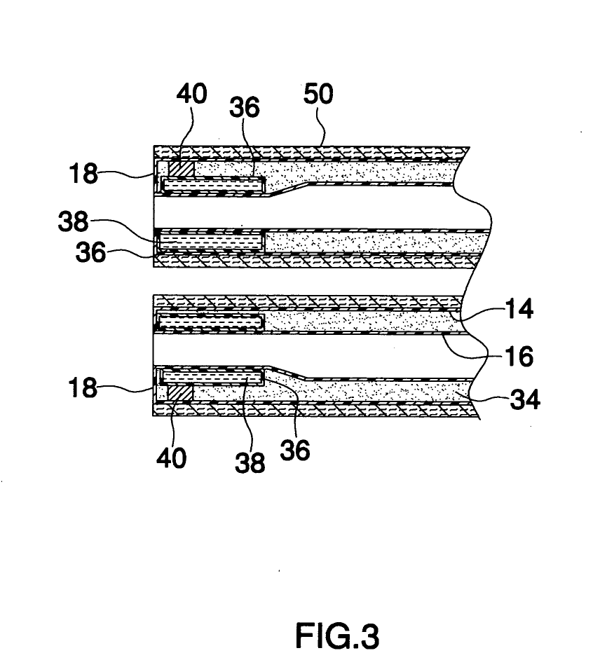

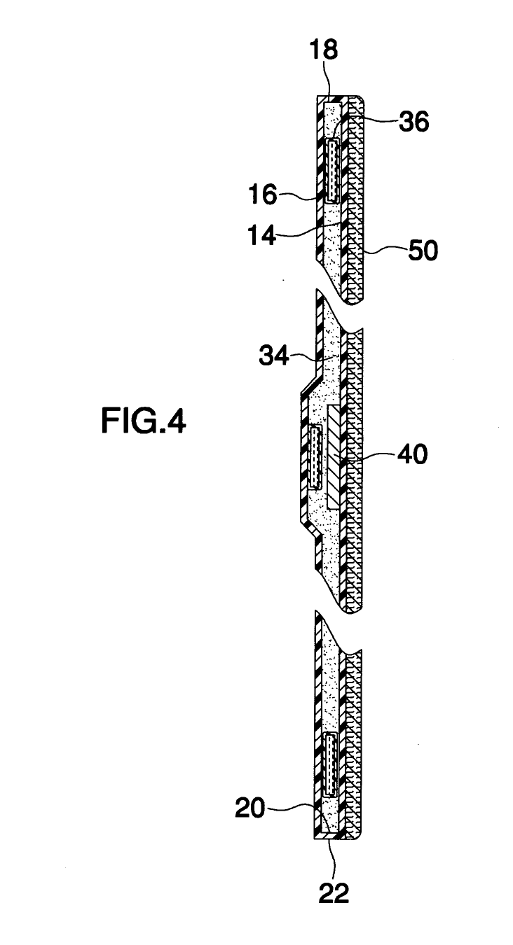

With reference now to the drawings, and in particular to FIGS. 1 through 4 thereof, a new cooling device embodying the principles and concepts of the present invention and generally designated by the reference numeral 10 will be described.

As best illustrated in FIGS. 1 through 4, the cooling blanket assembly 10 generally comprises a substantially watertight housing 12 having a top wall 14, a bottom wall 16 and a peripheral wall 18 extending between and attached to the top 14 and bottom 16 walls. The housing 12 is preferably constructed of a relatively thick and flexible plastic material. The housing 12 has an inner surface 20 and an outer surface 22. The housing 12 preferably has a substantially rectangular shape with a length generally between 3 feet and 5 feet, a width generally between 3 feet and 5 feet and a height generally between ½ inch and 2 inches. The peripheral wall 18 includes a first side wall 23, a second side wall 24, a third side wall 25 and a fourth side wall 26 wherein the first 23 and second 24 side walls are positioned opposite of each other. A cloth covering 50 may be attached to the top wall 14 so that it substantially covers the outer surface 22 of the top wall 14. The cloth covering 50 aids in the comfort of the user of the device.

An interior of the housing 12 is divided into a plurality of regions 30. Each of the regions 30 has a substantially equal size. Each of the regions 30 is fluidly isolated from each other. Preferably, three of the regions 30 are positioned adjacent to the first side wall 23 and three of the regions 30 are positioned adjacent to the second side wall 24 so that a total of six regions 30 are formed. The housing 12 can be folded along the lines 32 delineating the regions 30 without damaging or rupturing any contents of the housing 12. The lines 32 may be formed by areas where the top wall 14 is sealed to the bottom wall 16, or by dividing walls extending between the top 14 and bottom 16 walls.

A plurality of ammonium nitrate crystals 34 is positioned within the housing 12 such that each of the regions 30 has ammonium nitrate 34 therein. The amount of ammonium nitrate 34 is preferably between 3 ounces and 16 ounces per region, though this amount may vary depending upon the size of the housing 12.

Each of a plurality of containers 36 is positioned within the housing 12. Each of the containers 36 has water 38 therein. The amount of water 38 in each of the containers 36 is preferably between ¾ cup and 1¼ cup. The containers 36 are each positioned adjacent to the peripheral wall 18. Preferably, each of the containers 36 is positioned adjacent to one of the first 23 and second 24 side walls and each of the regions 30 has one of the containers 36 therein. The containers 36 are each elongated and each has a longitudinal axis orientated perpendicular to the first side wall 23. The containers 36 are preferably comprised of a relatively thin, plastic material which may be flexible or rigid. The containers 36 are attached to the lining 28 so that they are not movable within the housing 12.

Each of a pair of rigid plates 40 is attached to the inner surface 20 of the top wall 14. Each of the plates 40 is positioned adjacent to one of the first 23 and second 24 side walls and are positioned generally opposite of each other. The plates 40 are each elongated and each has a longitudinal axis orientated parallel to the first side wall 23. The plates 40 are preferably comprised of a relatively thick plastic material or a metallic material. The plates are generally centered within the housing 12 so that a line bisecting the first 23 and second 24 side walls also extends through each of the plates.

In use, when a person is suffering from hyperthermia, the containers 36 may be broken on the plates 40 so that the water 38 contained therein saturates adjacently positioned portions of the ammonium nitrate 34. The subsequent reaction between the ammonium nitrate 34 and the water 38 produces an endothermic reaction which cools the housing 12 and draws heat away from the person's body. The positioning of the containers 36 ensures that they are easily located for breakage. The orientation of the plates 40, with respect to the containers 36, allows for easier breakage of the containers 36 by bending them around the plates 40. The housing may be folded as shown in FIG. 1 so that the plates 40 are aligned with each of the containers 38. As user of the assembly may step on the upper plate to break open each of the containers 38 positioned therebetween. This allows for quick mixing of the ammonium nitrate 34 and water 38.

With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1. A cooling blanket assembly comprising:
a substantially waterproof housing having a top wall, a bottom wall and a peripheral wall extending between and attached to said top and bottom walls, said housing having an inner surface an outer surface, said housing having a substantially rectangular shape, said housing having a length generally between 3 feet and 5 feet and a width generally between 3 feet and 5 feet, said housing comprising a flexible material;
a plurality of ammonium nitrate crystals being positioned within said housing;
a plurality of containers being positioned within said housing, each of said containers having water therein;
a pair of rigid plates, each of said rigid plates being attached to said inner surface of said top wall; and
wherein said containers may be selectively broken on said plates such that the water contained therein saturates adjacently positioned portions of the ammonium nitrate.

2. The assembly according to claim 1, wherein an interior of said housing is divided into a plurality of regions, each of said regions having a substantially equal size, each of said regions being fluidly isolated from each other, each of said regions having ammonium nitrate therein, each of said regions having one of said containers therein.

3. The assembly according to claim 2, wherein said peripheral wall includes a first side wall, a second side wall, a third side wall and a fourth side wall wherein said first and second side walls are positioned opposite of each other, three of said regions being positioned adjacent to said first side wall and three of said regions being positioned adjacent to said second side wall.

4. The assembly according to claim 1, wherein each of said containers are positioned adjacent to said peripheral wall.

5. The assembly according to claim 1, wherein said peripheral wall includes a first side wall, a second side wall, a third side wall and a fourth side wall wherein said first and second side walls are positioned opposite of each other, each of said containers being positioned adjacent to one of said first and second side walls, each of said plates being positioned adjacent to one of said first and second side walls.

6. The assembly according to claim 5, wherein each of said containers is elongated, each of said containers having a longitudinal axis orientated perpendicular to said first side wall, each of said plates being elongated and having a longitudinal axis orientated parallel to said first side wall.

7. The assembly according to claim 1, wherein each of said containers is elongated, each of said containers having a longitudinal axis orientated perpendicular to said first side wall, each of said plates being elongated and having a longitudinal axis orientated parallel to said first side wall.

8. The assembly according to claim 4, wherein each of said plates is positioned adjacent to said peripheral wall, each of said containers is elongated, each of said containers having a longitudinal axis orientated perpendicular to said first side wall, each of said plates being elongated and having a longitudinal axis orientated parallel to said first side wall.

9. A cooling blanket assembly comprising:
a substantially waterproof housing having a top wall, a bottom wall and a peripheral wall extending between and attached to said top and bottom walls, said housing having an inner surface an outer surface, said housing having a substantially rectangular shape, said housing having a length generally between 3 feet and 5 feet, a width generally between 3 feet and 5 feet and a height generally between ½ inch and 2 inches, said peripheral wall including a first side wall, a second side wall, a third side wall and a fourth side wall wherein said first and second side walls are positioned opposite of each other, an interior of said housing being divided into a plurality of regions, each of said regions having a substantially equal size, each of said regions being fluidly isolated from each other, three of said regions being positioned adjacent to said first side wall and three of said regions being positioned adjacent to said second side wall, said housing comprising a flexible material;
a plurality of ammonium nitrate crystals being positioned within said housing such that each of said regions has ammonium nitrate therein;
a plurality of containers being positioned within said housing, each of said containers having water therein, each of said containers being positioned adjacent to said peripheral wall, each of said containers being positioned adjacent to one of said first and second side walls, each of said regions having one of said containers therein, each of said containers being elongated, each of said containers having a longitudinal axis orientated perpendicular to said first side wall;
a pair of rigid plates, each of said rigid plates being attached to said inner surface of said top wall, each of said plates being positioned adjacent to one of said first and second side walls, said plates being positioned generally opposite of each other, each of said plates being elongated and having a longitudinal axis orientated parallel to said first side wall, said plates being positioned such that a line bisecting each of said first and second side walls extends through each of said plates; and
wherein said containers may be selectively broken on said plates such that the water contained therein saturates adjacently positioned portions of the ammonium nitrate.

10. The assembly according to claim 9, further including a cloth material being attached to and covering said top wall.
